(19) United States
(12) Patent Application Publication
(10) Pub. No.: US 2026/0263707 A1
(43) Pub. Date: Sep. 10, 2026

(54) NEBULIZER DISPLAY SYSTEM

(71) Applicant: HCMed Innovations Co., LTD., Taipei City (TW)

(72) Inventor: CHUN-YING LI, New Taipei City (TW)

(21) Appl. No.: 19/554,671

(22) Filed: Mar. 3, 2026

Related U.S. Application Data
(60) Provisional application No. 63/766,381, filed on Mar. 4, 2025.

(30) Foreign Application Priority Data
Mar. 2, 2026 (CN) 202610243554.7

(51) Int. Cl. A61M 11/00; A61M 15/00

(52) U.S. Cl. CPC A61M 11/003; A61M 11/005; A61M 15/008; A61M 2202/0007; A61M 2202/04; A61M 2205/3327; A61M 2205/3592; A61M 2205/502; A61M 2205/583; A61M 2205/6009; A61M 2205/8206

(57) ABSTRACT

A nebulizer display system includes a display device and a nebulizer. The display device is configured to maintain an outputted visual record when not powered, and the display device includes an authentication code carrier. The nebulizer is configured to detect drug administration information related to a nebulized medicine sprayed by the nebulizer, and establish a connection with the authentication code carrier when sensing the authentication code carrier to transmit the drug administration information to the authentication code carrier. The authentication code carrier induces an induced current through an induction effect of the nebulizer to supply power to the display device. The display device updates the outputted visual record based on the drug administration information received by the authentication code carrier.

### CROSS-REFERENCE TO RELATED PATENT APPLICATION

This application claims the benefit of priority to China Patent Application No. 202610243554.7, filed on Mar. 2, 2026, and the U.S. Provisional Patent Application Ser. No. 63/766381, filed on Mar. 4, 2025, which application is incorporated herein by reference in its entirety.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to a nebulizer technology, and more particularly to a nebulizer display system.

### BACKGROUND OF THE DISCLOSURE

Existing portable nebulizers (e.g., medicinal inhalation nebulizers) still have several problems in use. Specifically, during a treatment process, users often rely solely on whether the medicine liquid has been completely nebulized and sprayed as a basis for completing the treatment process, making it difficult to grasp drug administration information in real time, and causing significant inconvenience in managing medication adherence.

In addition, existing nebulizers generally lack functions of medicine authentication and user identity authentication. This deficiency creates risks of medication misuse, leading to unintended use by non-target users such as to cause physical discomfort.

Accordingly, how to improve the management of medication adherence and solve problems caused by the lack of medicine authentication and identity authentication has become an urgent task for those skilled in the art.

### SUMMARY OF THE DISCLOSURE

In response to the above-referenced technical inadequacies, the present disclosure provides a nebulizer display system.

In order to solve the above-mentioned problems, one of the technical aspects adopted by the present disclosure is to provide a nebulizer display system. The nebulizer display system includes a display device and a nebulizer. The display device is configured to maintain an outputted visual record when not powered, and the display device includes an authentication code carrier. The nebulizer is configured to detect drug administration information related to a nebulized medicine sprayed by the nebulizer, and establish a connection with the authentication code carrier when sensing the authentication code carrier, so as to transmit the drug administration information to the authentication code carrier. The authentication code carrier induces an induced current through an induction effect of the nebulizer to supply power to the display device. The display device updates the outputted visual record based on the drug administration information received by the authentication code carrier.

In conclusion, the nebulizer display system provided by the present disclosure utilizes the display device that maintains the outputted visual record even in a non-powered state to continuously display medication status of a current treatment phase. This feature enables a user to instantly grasp drug usage information even when the device is not powered, avoiding medication at the wrong time due to missing information, thereby improving treatment safety and adherence. In addition, the system integrates mechanisms of medicine authentication and identity authentication. The mechanism of the medicine authentication compares a medicine authentication code with a medicine-specific database to ensure that the used nebulized medicine is correct. The identity authentication mechanism confirms identification of a treatment target through biometrics or login credentials to avoid misuse of the nebulized medicine by an non-target user. Further, the system also stores usage records in a built-in memory or a cloud database for subsequent analysis by medical personnel to establish a complete treatment tracking file. In this way, the system comprehensively improves safety, precision, and traceability of drug treatment, providing a more efficient and reliable solution for clinical applications and home care.

These and other aspects of the present disclosure will become apparent from the following description of the embodiment taken in conjunction with the following drawings and their captions, although variations and modifications therein may be effected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a functional block diagram of a nebulizer display system according to an embodiment of the present disclosure;

FIG. 2A is a functional block diagram of the display device according to some exemplary embodiments of the present disclosure;

FIG. 2B is a schematic view of installation of an authentication code carrier on a display device according to some exemplary embodiments of the present disclosure;

FIG. 3 is a functional block diagram of the display device according to other exemplary embodiments of the present disclosure;

FIG. 4 is a functional block diagram of the display device according to other exemplary embodiments of the present disclosure;

FIG. 5 is a schematic view of authentication performed on a nebulizer according to some exemplary embodiments of the present disclosure; and

FIG. 6 is a flowchart of a nebulizer display method according to some exemplary embodiments of the present disclosure.

### DETAILED DESCRIPTION OF THE EXEMPLARY EMBODIMENTS

The present disclosure is more particularly described in the following embodiments and examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a,” “an” and “the” includes plural reference, and the meaning of “in” includes “in” and “on.” Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first,” “second” or “third” can be used to describe various components, signals or the like, which are for distinguishing one component/signal from another one only, and are not intended to, nor should be construed to impose any substantive limitations on the components, signals or the like.

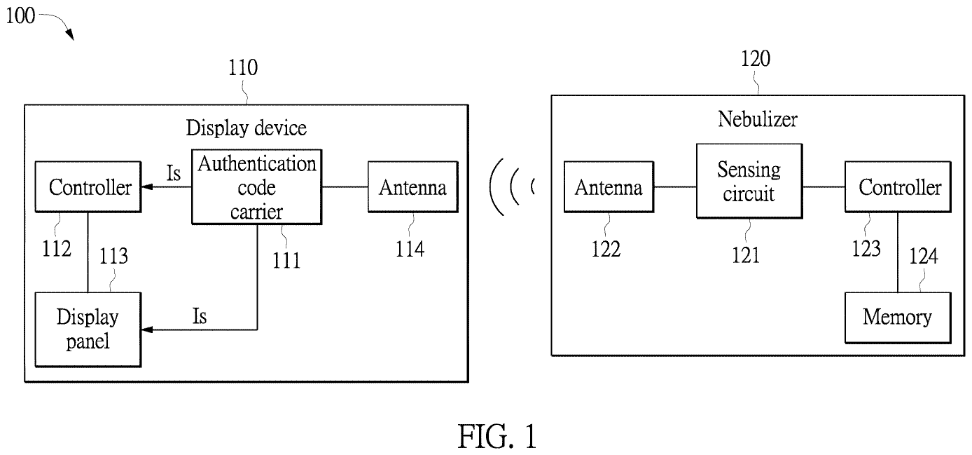

Referring to FIG. 1, FIG. 1 is a functional block diagram of a nebulizer display system 100 according to an embodiment of the present disclosure. As shown in FIG. 1, in this embodiment, the nebulizer display system 100 includes a display device 110 and a nebulizer 120.

In some embodiments, the display device 110 is detachably installed on the nebulizer 120 through manners such as snap-fit fastening, magnetic attraction, or the like.

In this embodiment, the display device 110 is used to maintain an outputted visual record when not powered. The display device 110 includes an authentication code carrier 111.

In some embodiments, the display device 110 is implemented by any flexible display (e.g., electronic paper, electronic book, electronic notebook, organic light-emitting diode (OLED) display, or the like) that maintains the outputted visual record based on electrophoretic display (EPD) technology. In some embodiments, the display device 110 first fixes charged particles used for development at a specific position when powered, so as to maintain the outputted visual record when not powered.

In some embodiments, the authentication code carrier 111 is fixedly (i.e., non-detachably) disposed in the display device 110. In some embodiments, the authentication code carrier 111 is implemented by any sensing chip (e.g., a near-field communication (NFC) tag chip or a radio-frequency identification (RFID) tag chip) having a passive proximity sensing function (e.g., a passive mode function of NFC or RFID).

In this embodiment, the nebulizer 120 is used to detect drug administration information related to a nebulized medicine sprayed by the nebulizer 120, and establish a connection with the authentication code carrier 111 when sensing the authentication code carrier 111, so as to transmit the drug administration information to the authentication code carrier 111.

Next, the authentication code carrier 111 induces an induced current Is through the induction effect of the nebulizer 120 to supply power to the display device 110. Next, the display device 110 updates the outputted visual record based on the drug administration information received by the authentication code carrier 111.

In some embodiments, the nebulizer 120 is implemented by any nebulizer (e.g., a mesh nebulizer) used to spray the nebulized medicine. In some embodiments, the nebulized medicine is implemented by any inhaled medicine (e.g., a bronchodilator, a glucocorticoid agent, or the like) processed by vibration nebulization.

In some embodiments, the drug administration information includes authentication information of the nebulized medicine and medication adherence information of a user. The authentication information includes a medicine name, a pharmaceutical company name, and a medicine authentication code. The medication adherence information includes a remaining medication dose count, an administration time, a treatment time, an administration ratio, a user respiratory parameter, and the like, but is not limited thereto.

In detail, the medicine name is a name used to identify ingredients and usage of the nebulized medicine. The pharmaceutical company name is a name indicating a source (i.e., a manufacturer or a company that produces or sells the nebulized medicine) of the medicine. The medicine authentication code is a medicine license number or an authentication code issued by a medical authority (e.g., the U.S. Food and Drug Administration) to track medicine information.

In addition, the remaining medication dose count indicates how many more times the nebulized medicine can be inhaled by the user. The administration time indicates a time point when a plurality of treatment phases (e.g., a plurality of time periods preset to require the use of the nebulizer 120) start. The treatment time indicates a total duration of a single treatment phase. The administration ratio indicates a ratio of the nebulized medicine actually inhaled in the single treatment phase. The user respiratory parameter includes a pressure value and an airflow value. The pressure value indicates pressure in the respiratory tract detected when the user inhales, and the airflow value indicates an air flow rate of inhalation and exhalation by the user.

It should be noted that subsequent paragraphs will explain how these data in the drug administration information are generated, and will not be repeated here.

In some embodiments, the display device 110 further includes a controller 112 and a display panel 113. The controller 112 is connected to the authentication code carrier 111 and the display panel 113, and the display panel 113 is connected to the authentication code carrier 111.

In some embodiments, the controller 112 is implemented by any control circuit (e.g., a microcontroller (MCU), a microprocessor unit (MPU), or the like) having a data processing function. In some embodiments, the display panel 113 is implemented by any panel (e.g., an electrophoretic display panel) directed to electrophoretic display technology.

In some embodiments, the authentication code carrier 111 uses the induced current Is to supply power to the controller 112 and the display panel 113, and transmit the drug administration information received from the nebulizer 120 to the controller 112. The controller 112 controls the display panel 113 to update the outputted visual record to display the drug administration information, so as to maintain the outputted visual record having the drug administration information when not powered.

In other embodiments, the outputted visual record displays a part of the drug administration information (i.e., only displays the respective administration times of a previous treatment phase and a current treatment phase, and the remaining medication dose count (e.g., displaying “Current administration time: 13:30”, “Next administration time: 19:30”, and “Remaining medication dose count: 26”)).

In some embodiments, the display device 110 further includes an antenna 114. In some embodiments, the nebulizer 120 includes a sensing circuit 121 and an antenna 122.

In some embodiments, the antenna 114 and the antenna 122 are implemented by any antenna structure (e.g., a microstrip antenna or a printed antenna, or the like) having a wireless communication function. In some embodiments, the sensing circuit 121 is implemented by any sensor (e.g., a NFC reader, a RFID reader, or the like) having an active proximity sensing function (e.g., an active mode function of NFC or RFID).

In some embodiments, when the sensing circuit 121 senses the authentication code carrier 111 through the antenna 114 by using the antenna 122, the nebulizer 120 establishes the connection with the authentication code carrier 111.

Specifically, the sensing circuit 121 actively generates a magnetic field through the antenna 122. When the authentication code carrier 111 enters this magnetic field, an induced current Is (i.e., the induction effect of electromagnetism) is induced on the authentication code carrier 111 through the antenna 114, and the authentication code carrier 111 sends a reply message (e.g., including the authentication code of the authentication code carrier 111) back to the nebulizer 120 through the antenna 114.

When receiving the reply message, the nebulizer 120 identifies that the authentication code carrier 111 has been sensed. Next, the nebulizer 120 completes a communication protocol with the authentication code carrier 111 to establish the connection when authentication of the authentication code of the authentication code carrier 111 is completed (e.g., identifying that authentication code matches a tag authentication code pre-stored in the nebulizer 120).

In some embodiments, the nebulizer 120 further includes a controller 123 and a memory 124. The controller 123 is used to control the sensing circuit 121 to perform proximity sensing through the antenna 122 (i.e., the sensing circuit 121 senses the authentication code carrier 111 to perform the communication protocol when the authentication code carrier 111 is close to the sensing circuit 121). The memory 124 stores the detected drug administration information.

In some embodiments, the controller 123 is also implemented by any control circuit having the data processing function. In some embodiments, the memory 124 is implemented by any storage circuit (e.g., a storage unit, a flash memory, or the like) having a data storage function.

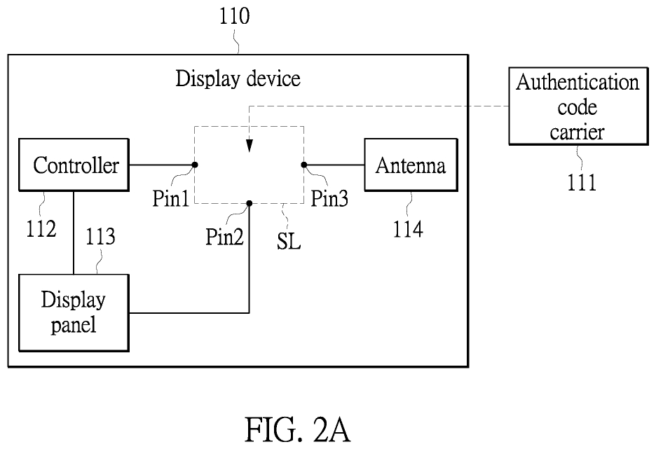

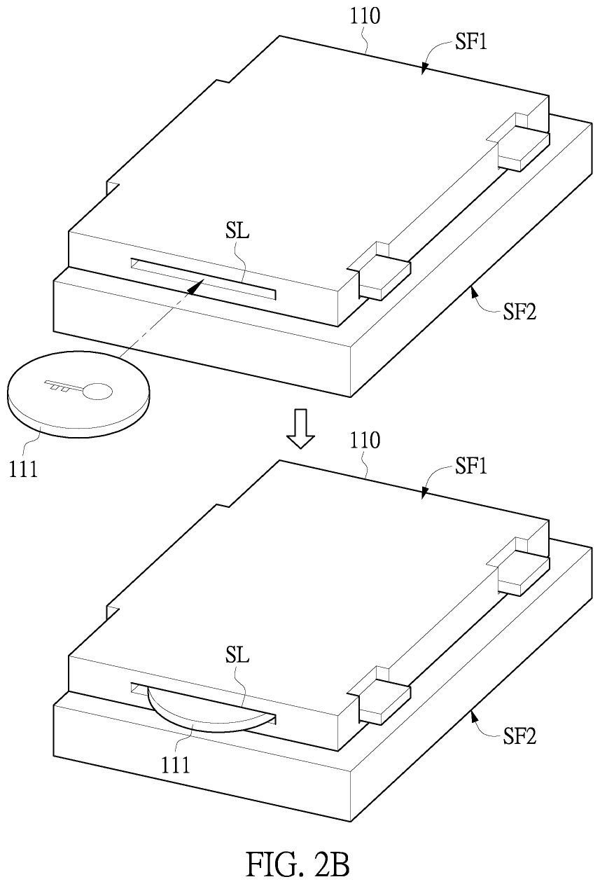

Referring to FIG. 2A, FIG. 2A is a functional block diagram of the display device 110 according to some exemplary embodiments of the present disclosure. As shown in FIG. 2A, in this embodiment, the difference between the display device 110 of this embodiment and FIG. 1 is that the authentication code carrier 111 is detachably disposed in the display device 110 and electrically connected to the controller 112, the display panel 113, and the antenna 114 through a plurality of electrical contacts Pin1-Pin3, while other similarities are not repeated here.

Specifically, the display device 110 has a slot SL, which is used for inserting the authentication code carrier 111. When the authentication code carrier 111 is inserted into the slot SL, the authentication code carrier 111 is electrically connected to the controller 112, the display panel 113, and the antenna 114 through the electrical contacts Pin1-Pin3 in the slot SL. In this way, the user needs to go to a pharmacy or a point of sale of the nebulizer 120 when the remaining medication dose count of the detachable authentication code carrier 111 is 0, so as to update the remaining medication dose count of a new authentication code carrier 111 to an initial medication dose count, before the nebulizer 120 is used again.

Referring to FIG. 2B, FIG. 2B is a schematic view of installation of an authentication code carrier 111 on a display device 110 according to some exemplary embodiments of the present disclosure. As shown in FIG. 2B, in this embodiment, the display device 110 has a first surface SF1 and a second surface SF2 that are opposite to each other. The slot SL is opened above the authentication code carrier 111 at a position adjacent to the first surface SF1 for the installation of the authentication code carrier 111.

In some embodiments, the display panel 113 and the antenna 114 are disposed on the second surface SF2. In this way, such an arrangement of the antenna 114 maintains an original induction area of the antenna 114, without causing alignment problems during use due to the limitation of the induction area of the antenna 114 by the authentication code carrier 111.

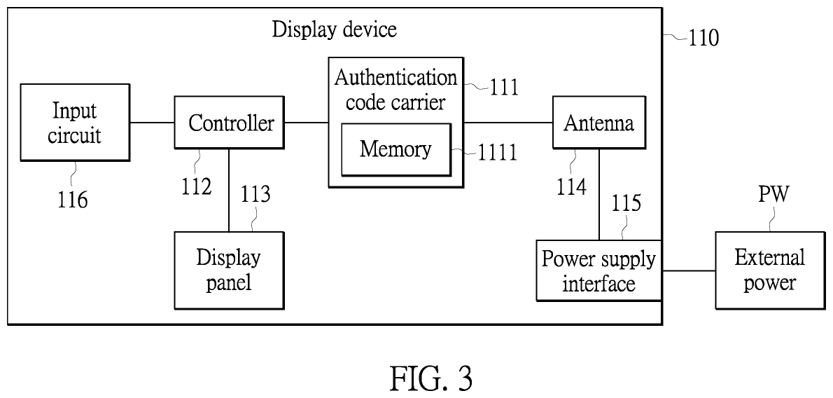

Referring to FIG. 3, FIG. 3 is a functional block diagram of the display device 110 according to other exemplary embodiments of the present disclosure. As shown in FIG. 3, in this embodiment, the difference between this display device 110 of this embodiment and FIG. 1 is that the authentication code carrier 111 includes a memory 1111, the display device 110 further includes a power supply interface 115, and the authentication code carrier 111 is not connected to the display panel 113, while other similarities are not repeated here.

Specifically, the memory 1111 stores the drug administration information (i.e., the drug administration information previously received from the nebulizer 120). In the absence of the induced current Is, the power supply interface 115 supplies power to the display device 110 from an external power PW (e.g., utility power). That is, when the authentication code carrier 111 is not connected to the nebulizer 120 (at which time there is no induced current Is within the display device 110), the controller 112 still accesses the drug administration information in the memory 1111 and controls the display panel 113 to display the drug administration information due to power supplied by the external power PW.

In some embodiments, the power supply interface 115 is implemented by any interface (e.g., a universal serial bus (USB) interface or a wireless power transfer interface) capable of obtaining power from the external power PW. In some embodiments, the memory 1111 is also implemented by any storage circuit (e.g., a storage unit or a flash memory) having the data storage function.

In some embodiments, the controller 112 records each received drug administration information as historical information in the memory 1111. The historical information includes historical authentication information (including all authentication information received during all treatment phases prior to the current treatment phase) and historical medication adherence information (including all medication adherence information received during all treatment phases prior to the current treatment phase).

In some embodiments, the display device 110 further includes an input circuit 116. The input circuit 116 is used for the user to select the authentication information and the medication adherence information of one of the treatment phases for display on the display panel 113. In some embodiments, the input circuit 116 is implemented by any circuit (e.g., a button input circuit or a touch input circuit) having an input function.

By the external power PW supplying power to the display device 110 through the power supply interface 115, the display device 110 saves space for installing a battery, thereby significantly reducing the volume and thickness of the display device 110.

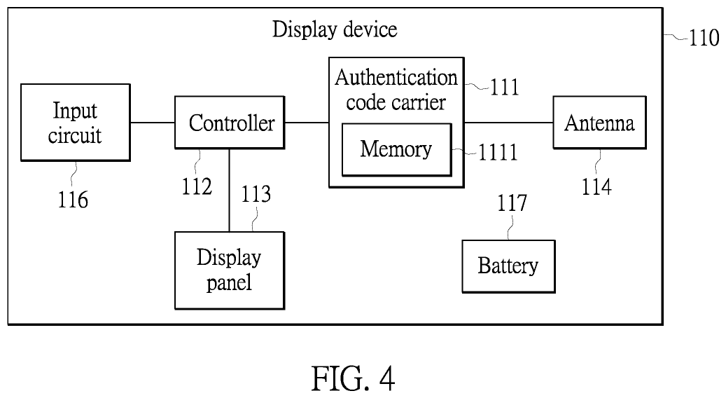

Referring to FIG. 4, FIG. 4 is a functional block diagram of the display device 110 according to other exemplary embodiments of the present disclosure. As shown in FIG. 4, in this embodiment, the difference between this display device 110 of this embodiment and FIG. 3 is that the display device 110 further includes a battery 117 to supply power instead of the power supply interface 115 in FIG. 3, while other similarities are not repeated here.

Specifically, in the absence of the induced current Is, the battery 117 is used to supply power to the display device 110. That is, when the authentication code carrier 111 is not connected to the nebulizer 120 (at which time there is no induced current Is within the display device 110), the controller 112 still accesses the drug administration information in the memory 1111 and controls the display panel 113 to display the drug administration information due to the power supplied by the battery 117.

By the battery 117 supplying power to the display device 110, the display device 110 no longer needs to be connected to an external power PW, thereby significantly improving the portability of the display device 110.

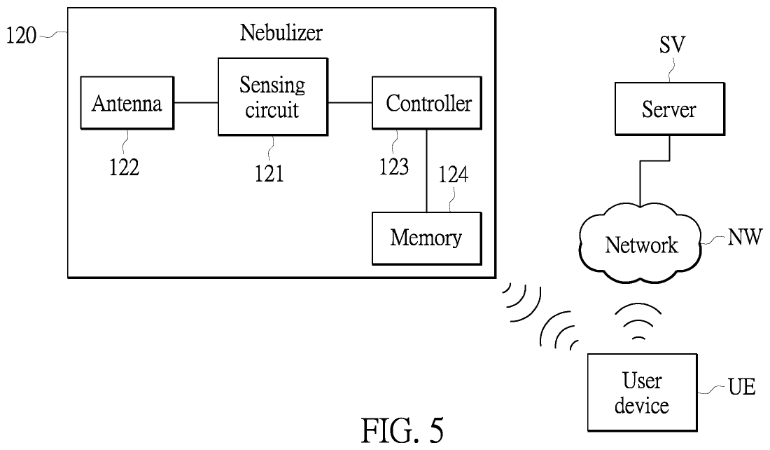

Referring to FIG. 5, FIG. 5 is a schematic view of authentication performed on the nebulizer 120 according to some exemplary embodiments of the present disclosure. As shown in FIG. 5, in this embodiment, the nebulizer 120 performs various authentications or data uploads to an external server SV (e.g., a cloud server or a distributed server managed by a manufacturer of the nebulizer 120) through a user device UE (e.g., a portable device such as a smartphone, a smart watch, a smart bracelet, a smart ring, or an item tracker (e.g., AirTag)) that is pre-connected (i.e., paired) to the nebulizer 120 and an external network NW.

In some embodiments, the nebulizer 120 performs medicine authentication and obtains the authentication information of the nebulized medicine from the server SV through the paired user device UE.

In some embodiments, when sensing an authentication code carrier (not shown) of the nebulized medicine built in a medicine bottle or a medicine box (not shown), the nebulizer 120 accesses the medicine authentication code (e.g., a Unique Identifier (UID)) and the current remaining medication dose count in the authentication code carrier of the nebulized medicine.

Next, when the remaining medication dose count is greater than zero, the nebulizer 120 uploads the medicine authentication code to the server SV through the user device UE to make server SV authenticate the medicine authentication code (e.g., checking whether the medicine authentication code complies with official anti-counterfeiting standards or using a preset key to authenticate authenticity). By contrast, when the remaining medication dose count is equal to zero, the nebulizer 120 is locked (i.e., unable to spray the nebulized medicine) and forcefully deactivates the authentication code carrier of the nebulized medicine to prevent refilling or excessive medication. At this time, the user needs to go to the pharmacy or the point of sale of the nebulizer 120 to obtain a new medicine bottle or a new medicine box having a new authentication code carrier before the nebulized medicine is used again.

Next, when the authentication is successful, the nebulizer 120 is unlocked and activated, and the remaining medication dose count is decreased (i.e., decreased by one) to write the updated remaining medication dose count (which serves as a part of the drug administration information) back to the authentication code carrier of the nebulized medicine (ensuring that the remaining medication dose count is accurately transitioned even if switched to a different nebulizer). By contrast, when the authentication fails, the nebulizer 120 is also locked.

In some embodiments, whenever establishing the connection with the authentication code carrier 111, the nebulizer 120 also first checks whether the remaining medication dose count of the authentication code carrier 111 is zero, and performs locking when the remaining medication dose count of the authentication code carrier 111 is zero. At this time, the user needs to go to the pharmacy or the point of sale of the nebulizer 120 to obtain a new display device or update the remaining medication dose count of the authentication code carrier 111 before the nebulizer 120 is used again.

In some embodiments, when establishing the connection with the authentication code carrier 111, the nebulizer 120 also writes the updated remaining medication dose count into the authentication code carrier 111 of the display device 110 as the remaining medication dose count of the authentication code carrier 111.

In some embodiments, when the authentication is successful, the server SV transmits the authentication information (including the medicine authentication code, and the medicine name and the pharmaceutical company name corresponding to the medicine authentication code) to the user device UE.

In some embodiments, different nebulized medicines correspond to different initial medication dose counts (the initial medication dose count of the nebulized medicine is equal to an initial medication dose count of the authentication code carrier 111), and the initial medication dose count indicates a total number of times that the nebulized medicine can be inhaled in all treatment phases.

In some embodiments, the nebulizer 120 measures an administered dose count of the nebulized medicine to generate medication adherence information of the nebulized medicine. Next, the nebulizer 120 generates the drug administration information including the authentication information and the medication adherence information.

In some embodiments, the nebulizer 120 further includes various sensors (not shown). These sensors are used to detect the administration time, the treatment time, the administration ratio, and the user respiratory parameter during each treatment phase when the nebulizer 120 is used. The sensors are, for example, a flow meter, a conductivity sensor for medicinal liquid concentration, or a pressure sensor.

In some embodiments, when unlocking is successful and a negative pressure is detected (e.g., the pressure sensor detects user inhalation), the nebulizer 120 activates, for example, a piezoelectric ceramic or an ultrasonic vibration module (not shown) to nebulize the medicine and start spraying. This continues until a single preset dose (e.g., 50 ml) is completely sprayed or a preset time (e.g., 2 minutes) is reached.

In some embodiments, when the nebulizer 120 finishes spraying the nebulized medicine, the controller 123 uses the updated remaining medication dose count, as well as the administration time, the treatment time, the administration ratio, and the user respiratory parameter detected in the current treatment phase as the medication adherence information.

In some embodiments, in one of the treatment phases, the nebulizer 120 performs identity authentication on the user using the nebulizer 120 through the user device UE paired with the nebulizer 120. The nebulizer 120 starts to spray the nebulized medicine only when the identity authentication is successful and the authentication information is successfully authenticated.

In some embodiments, the user device UE pre-stores user information. When the user inputs the identity information into the user device UE, the user device UE compares the user information with the identity information.

When the user information matches the identity information, the user device UE unlocks the nebulizer 120. By contrast, when the user information does not match the identity information, the user device UE locks the nebulizer 120.

In other embodiments, the user information is also stored in the server SV. At this time, the user device UE completes the comparison between the user information and the inputted identity information through the server SV.

In some embodiments, the user information is biometrics (e.g., fingerprints, iris, or voiceprints) of the user or login credentials (e.g., a preset account and password). The identity information is the biometrics or the login credentials inputted on the user device UE.

In some embodiments, each time the nebulizer 120 generates the drug administration information, the nebulizer 120 instantly uploads the drug administration information to the server SV through the user device UE. In this way, medical personnel can download the drug administration information from the server SV for viewing, significantly increasing the convenience of managing medication adherence.

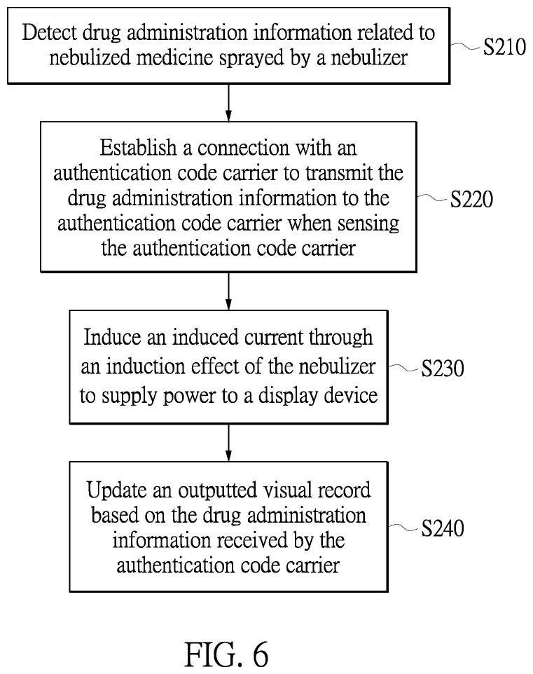

Referring to FIG. 6, FIG. 6 is a flowchart of a nebulizer display method according to some exemplary embodiments of the present disclosure. The nebulizer display method is applicable to the nebulizer display system 100 shown in FIG. 1. As shown in FIG. 6, in this embodiment, the nebulizer display method includes steps S210 to S240.

First, in step S210, the nebulizer 120 detects the drug administration information related to the nebulized medicine sprayed by the nebulizer 120. In step S220, the nebulizer 120 establishes the connection with the authentication code carrier 111 to transmit the drug administration information to the authentication code carrier 111 when sensing the authentication code carrier 111.

In step S230, the authentication code carrier 111 induces an induced current Is through the induction effect of the nebulizer 120 to supply power to the display device 110. In step S240, the display device 110 updates the outputted visual record based on the drug administration information received by the authentication code carrier 111.

It should be noted that detailed operations of these steps have been described in detail in the above paragraphs and are not further repeated here.

### Beneficial Effects of the Embodiments

In conclusion, the nebulizer display system provided by the present disclosure utilizes the display device that maintains the outputted visual record even in a non-powered state to continuously display medication status of a current treatment phase. This feature enables a user to instantly grasp drug usage information even when the device is not powered, avoiding medication at the wrong time due to missing information, thereby improving treatment safety and adherence. In addition, the system integrates mechanisms of medicine authentication and identity authentication. The mechanism of the medicine authentication compares a medicine authentication code with a medicine-specific database to ensure that the used nebulized medicine is correct. The identity authentication mechanism confirms identification of a treatment target through biometrics or login credentials to avoid misuse of the nebulized medicine by an non-target user. Further, the system also stores usage records in a built-in memory or a cloud database for subsequent analysis by medical personnel to establish a complete treatment tracking file. In this way, the system comprehensively improves safety, precision, and traceability of drug treatment, providing a more efficient and reliable solution for clinical applications and home care.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A nebulizer display system, comprising:
a display device configured to maintain an outputted visual record when not powered, wherein the display device includes an authentication code carrier; and
a nebulizer configured to detect drug administration information related to a nebulized medicine sprayed by the nebulizer, and establish a connection with the authentication code carrier when sensing the authentication code carrier, so as to transmit the drug administration information to the authentication code carrier;
wherein the authentication code carrier induces an induced current through an induction effect of the nebulizer to supply power to the display device;
wherein the display device updates the outputted visual record based on the drug administration information received by the authentication code carrier.

2. The nebulizer display system according to claim 1, wherein:
the display device further includes a display panel and a controller;
wherein the display panel and the controller are connected to the authentication code carrier;
wherein the authentication code carrier uses the induced current to supply power to the display panel and the controller, and transmits the drug administration information to the controller; and
wherein the controller controls the display panel to update the outputted visual record to display the drug administration information.

3. The nebulizer display system according to claim 2, wherein the authentication code carrier is detachably disposed in the display device, and is electrically connected to the display panel and the controller through a plurality of electrical contacts.

4. The nebulizer display system according to claim 1, wherein:
the display device further includes a first antenna;
wherein the nebulizer includes a sensing circuit and a second antenna;
wherein the nebulizer establishes a connection with the authentication code carrier when the sensing circuit senses the authentication code carrier through the first antenna by using the second antenna.

5. The nebulizer display system according to claim 1, wherein:
the authentication code carrier includes a memory;
wherein the memory is configured to store the drug administration information;
wherein the display device further includes a power supply interface, a controller, and a display panel;
wherein the power supply interface is configured to supply power from an external power source to the display device; and
wherein the controller is configured to access the drug administration information in the memory and control the display panel to display the drug administration information when the authentication code carrier is not connected to the nebulizer.

6. The nebulizer display system according to claim 1, wherein:
the authentication code carrier includes a memory;
wherein the memory is configured to store the drug administration information;
wherein the display device further includes a battery, a controller, and a display panel;
wherein the battery is configured to supply power to the display device; and
wherein the controller is configured to access the drug administration information in the memory and control the display panel to display the drug administration information when the authentication code carrier is not connected to the nebulizer.

7. The nebulizer display system according to claim 1, wherein:
the drug administration information includes authentication information of the nebulized medicine and medication adherence information of a user;
wherein the authentication information includes a medicine name, a pharmaceutical company name, and a medicine authentication code;
wherein the medication adherence information includes a remaining medication dose count, an administration time, a treatment time, an administration ratio, and a user respiratory parameter.

8. The nebulizer display system according to claim 1, wherein the nebulizer is further configured to execute following steps:
performing medicine authentication and obtaining authentication information of the nebulized medicine from an external server through a user device paired with the nebulizer.

9. The nebulizer display system according to claim 8, wherein the nebulizer is further configured to execute following steps:
measuring an administered dose count of the nebulized medicine to generate medication adherence information of the nebulized medicine; and
generating the drug administration information including the authentication information and the medication adherence information.

10. The nebulizer display system according to claim 8, wherein the nebulizer is further configured to execute following steps:
performing identity authentication on a user using the nebulizer through a user device paired with the nebulizer; and
starting to spray the nebulized medicine when the identity authentication is successful and the authentication information is successfully authenticated.
